(19) United States
(12) Patent Application Publication
(10) Pub. No.: US 2020/0268353 A1
(43) Pub. Date: Aug. 27, 2020

(54) ULTRASOUND 3D FULL-BODY TOMOGRAPHY SYSTEM

(71) Applicant: Yuan-Ping HWANG, Hualien County (TW)

(72) Inventor: Yuan-Ping HWANG, Hualien County (TW)

(21) Appl. No.: 16/519,984

(22) Filed: Jul. 23, 2019

(30) Foreign Application Priority Data
Feb. 22, 2019 (TW) 108106082

(51) Int. Cl. A61B 8/08; G01S 7/52; G01S 15/89; A61B 8/14; A61B 8/00

(52) U.S. Cl. CPC A61B 8/483; G01S 7/52053; G01S 15/8993; A61B 8/4281; A61B 8/14; A61B 8/4488; G01S 7/52077

(57) ABSTRACT

The present invention provides an ultrasound three-dimensional (3D) full-body tomography system, comprising an ultrasound guided wave medium container, an ultrasound probe array, and an ultrasound tomography device. The ultrasound guided wave medium container has a detection space, wherein the detection space is filled with a guided wave medium in order for a subject's body to be immersed in the guided wave medium. The ultrasound probe array is provided in the ultrasound guided wave medium container and comprises a plurality of probe units, wherein the probe units are integrated into an annular array and are arranged along a periphery of the detection space. The ultrasound tomography device is for constructing a 3D image model based on data fed back from each pixel of the ultrasound probe array.

### BACKGROUND OF THE INVENTION

### 1. Technical Field

The present invention relates to a three-dimensional (3D) full-body imaging system and more particularly to a system that performs 3D full-body imaging by means of ultrasonic waves.

### 2. Description of Related Art

The rapid development of the biomedical industry has brought about significant progress in biomedical technology. For example, 3D images of an internal body part are generally obtained through nuclear magnetic resonance imaging, or MRI for short. A typical MRI process involves exposing a patient to a magnetic field, irradiating the patient with proper electromagnetic waves to alter the spin orientations, and thereby cause resonance, of the hydrogen atoms in the patient's body, and analyzing the electromagnetic waves produced by the hydrogen atoms. While MRI is less harmful to the human body than X rays and computed tomography (CT), the electromagnetic energy of the wide-angle radio-frequency emission used in the focusing or measuring step of MRI may turn into heat in a patient's body tissues, thereby raising the temperature of those tissues and subjecting the patient to potential injury.

As an alternative to MRI, ultrasound tomography causes no wounds, does not involve radiation, and has therefore found wide medical application, especially in obstetrics. Given that fetuses are sensitive to radiation, X rays and CT are seldom used to scan a pregnant woman or fetus; the ideal scanning method in such cases is to perform ultrasound tomography instead.

Ultrasound tomography has the following advantages over its conventional counterparts: 1) It is not radioactive and is safer than an X-ray, CT, or MRI scan; and 2) It provides real-time images, so not only can the time otherwise required for film development or digital imaging be saved, but also real-time monitoring can be achieved with ultrasound tomography (e.g., to determine blood flow velocity in the cardiovascular system to help identify the condition of a disease rapidly).

However, despite their advantageous feature of enabling rapid diagnosis, the existing ultrasound tomography detection methods produce only two-dimensional (2D) detection results and cannot be used in full-body imaging. Moreover, ultrasonic waves propagate poorly in certain media (e.g., bones) and therefore may not penetrate certain body parts; in other words, insufficient imaging may occur in some cases, one notable example of which is ultrasound tomography of the brain. Furthermore, the quality of ultrasound tomography can be poor when there is a large difference in acoustic impedance, e.g., when gas exists between the probe and the tissues to be scanned. Ultrasound tomography of the pancreas, for example, is made extremely difficult by the gas in the gastrointestinal tract. Similarly, ultrasound tomography of the lungs is impossible unless the target is pleural effusion or a tumor. Also, the limited probing depth of ultrasonic waves tends to hinder the imaging of body structures that are far from the skin, and this is especially true for those who are obese. All of the above may compromise the results of ultrasound medical examination.

### BRIEF SUMMARY OF THE INVENTION

In order to achieve the above objective, the present invention provides an ultrasound three-dimensional (3D) full-body tomography system, comprising an ultrasound guided wave medium container, an ultrasound probe array, and an ultrasound tomography device. The ultrasound guided wave medium container has a detection space, wherein the detection space is filled with a guided wave medium in order for a subject's body to be immersed in the guided wave medium. The ultrasound probe array is provided in the ultrasound guided wave medium container and comprises a plurality of probe units, wherein the probe units are integrated into an annular array and are arranged along a periphery of the detection space. The ultrasound tomography device is for constructing a 3D image model based on data fed back from each pixel of the ultrasound probe array.

Another objective of the present invention is to provide an ultrasound three-dimensional (3D) full-body tomography system, comprising an ultrasound guided wave medium container, a movable ultrasound probe assembly, and an ultrasound tomography device. The ultrasound guided wave medium container has a detection space, wherein the detection space is filled with a guided wave medium in order for a subject's body to be immersed in the guided wave medium. The movable ultrasound probe assembly comprises a linear carrier and an annular ultrasound probe array provided on the linear carrier, wherein the annular ultrasound probe array comprises a plurality of probe units integrated into an annular array and is movable along the detection space via the linear carrier. The ultrasound tomography device is for constructing a 3D image model based on a velocity of the linear carrier and on data fed back from the annular ultrasound probe array.

Comparing to the conventional techniques, the present invention has the following advantages:

1. The present invention uses an array of ultrasound probes to perform 3D full-body imaging on a patient, thereby enabling multidimensional image reconstruction for tissues that are impenetrable by ultrasonic waves. The prior art drawback of insufficient imaging is thus overcome.

2. According to the present invention, 3D full-body imaging is performed on a patient through an array of ultrasound probes to effectively prevent injury to the human body.

3. According to the present invention, the array of ultrasound probes can directly output 3D images of a patient or a body part, without having to convert 2D images into 3D ones in a subsequent step.

### BRIEF DESCRIPTION OF THE SEVERAL VIEWS OF THE DRAWINGS

FIG. 1 is a block diagram of the first embodiment of the present invention.

FIG. 2 is a schematic drawing of the first embodiment of the present invention.

FIG. 3 is a block diagram of the second embodiment of the present invention.

FIG. 4 is a schematic drawing of the second embodiment of the present invention.

### DETAILED DESCRIPTION OF THE INVENTION

The details and technical solution of the present invention are hereunder described with reference to accompanying drawings. For illustrative sake, the accompanying drawings are not drawn to scale. The accompanying drawings and the scale thereof are not restrictive of the present invention.

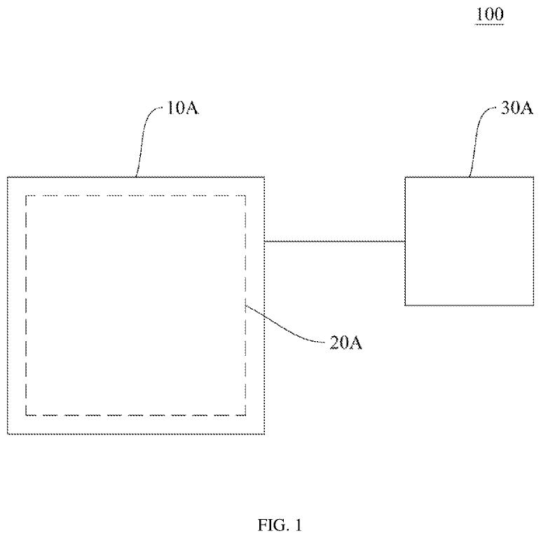

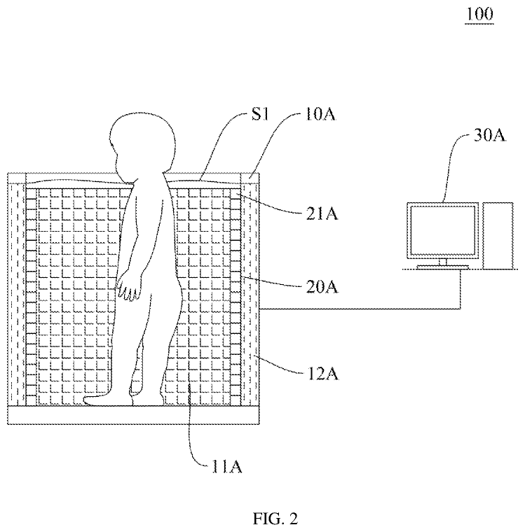

The technical content of the present invention is detailed below with reference to some illustrative embodiments of the invention. To start with, please refer to FIG. 1 and FIG. 2 respectively for a block diagram and a schematic drawing of the first embodiment of the invention.

The first embodiment discloses an ultrasound 3D full-body tomography system 100, which includes an ultrasound guided wave medium container 10A, an ultrasound probe array 20A, and an ultrasound tomography device 30A.

The ultrasound guided wave medium container 10A has a detection space S1. The detection space S1 is filled with a guided wave medium 11A so that a subject's body can be immersed in the guided wave medium 11A. In one preferred embodiment, the ultrasound guided wave medium container 10A is provided with an acoustic insulation layer 12A made of a sound-absorbing or noise-reducing material. As a barrier between the ultrasound probe array 20A and the environment outside the system, the acoustic insulation layer 12A may be provided anywhere on/in the ultrasound guided wave medium container 10A (e.g., on the outer or inner wall of the container or inside the container) as long as the acoustic insulation layer 12A corresponds in position to the ultrasound probe array 20A. To produce satisfactory detection results, the guided wave medium 11A may be water, degassed water, a developing agent, or a wave guiding gel; the present invention has no limitation in this regard.

The ultrasound probe array 20A is provided in the ultrasound guided wave medium container 10A and includes a plurality of probe units 21A, which are integrated into an annular array (the number of the probe units 21A being Lₜₕ*Hₜₕ) and are arranged along the periphery of the detection space S1.

In ultrasound medical examination, a phased array of piezoelectric transducers (generally made of ceramic) is typically used to generate short and strong acoustic pulses that form acoustic waves. Each probe unit 21A, therefore, includes a piezoelectric transducer packaged therein along with the related wires, in order for the ceramic transducer to oscillate when supplied with electrical pulses and thereby generate a series of acoustic pulses. The frequency of the resulting acoustic waves may be any frequency in the range of 1 to 13 THz and is hence far higher than those audible to human ears. The term “ultrasonic waves” as used herein refers to any acoustic wave whose frequency is higher than those able to be heard by human beings. The acoustic waves of the transducers will combine into a single acoustic wave that is focused and arcuate. The higher the frequency, the shorter the corresponding wavelength; and the shorter the wavelength, the higher the resolution of the image obtained. However, as the speed at which acoustic waves attenuate increases with the frequency of the acoustic waves, a relatively low frequency (3 to 5 THz) is preferable in order to probe tissues that are deep in the human body.

Each probe unit 21A is coated with rubber so that acoustic waves can propagate effectively into the subject (i.e., to achieve a match in impedance). The acoustic waves are partially reflected back to the probes by interfaces between different tissues, wherein the reflected acoustic waves are generally referred to as echoes. As is well known in the art, even tiny structures generate echoes (i.e., can reflect acoustic waves).

The paths taken by the echoes (i.e., the acoustic waves returning to, and to be received by, the probe units 21A) are similar to those of the acoustic waves emitted from the probe units 21A, except that the former paths and the latter paths run in opposite directions. The returning acoustic waves cause the transducers in the probe units 21A to oscillate, and the oscillation is converted into electrical pulses by the transducers. The probe units 21A send the electrical pulses to the ultrasound tomography device 30A in order for the ultrasound tomography device 30A to process the electrical pulses and thereby generate digital images.

The ultrasound tomography device 30A is an image processing device configured to construct a 3D image model based on the data fed back from each pixel (i.e., probe unit 21A) of the ultrasound probe array 20A. The ultrasound tomography device 30A receives three major types of parameters from the ultrasound probe array 20A: the location of each probe unit 21A that has received an echo (i.e., the location of each response-receiving pixel of the array), the signal intensity of each echo, and the flight times of ultrasonic waves (i.e., the response times).

Once the three types of data are obtained, the ultrasound tomography device 30A constructs a 3D model of the subject according to the data. Construction of the 3D image model may include applying time-division multiplexing (TDM) to the responses to the plural probe units 21A. The coordinates of each response-receiving pixel (i.e., the relative coordinates or world coordinates of the pixel in a 3D space) can be derived from the location of the corresponding response-receiving probe unit 21A and the corresponding flight time of ultrasonic waves. Moreover, in order to be mapped to a 3D space, the original images must be corrected in accordance with the locations of the probe units 21A while being converted into 3D images. For example, a reference point is set in the world coordinate system, and mapping computation is performed with reference to the reference point. Tissue densities in different areas can be derived from the signal intensities of the echoes and the flight times of ultrasonic waves, before the tissues are stratified depth-wise. The 3D images obtained can also be filtered using specific threshold values in order to produce images only of the area of interest (e.g., the blood system, the structure of a specific organ, tissues under pathological assessment, or a benign or malign tumor). The depth to which ultrasonic waves penetrate the subject (i.e., the sampling depth) can be changed by adjusting the power and frequency of the ultrasonic waves so that an image model can be constructed for a relatively shallow or relatively deep portion of the subject. In another preferred embodiment, the images obtained can be filled with different grayscale values or colors by setting specific threshold values, in order to accentuate the images of individual tissues.

Apart from the algorithms stated above, the present invention may use a single-input multi-output (SIMO) model, a multi-input single-output (MISO) model, or a multi-input multi-output (MIMO) model without limitation.

In this embodiment, the ultrasound probe array 20A surrounding the subject and the ultrasound tomography device 30A are configured on the assumption that the speed of sound is constantly 1540 m/s. While the echoes may lose some of the acoustic energy of the original acoustic waves, the loss is nominal when compared with attenuation caused by absorption.

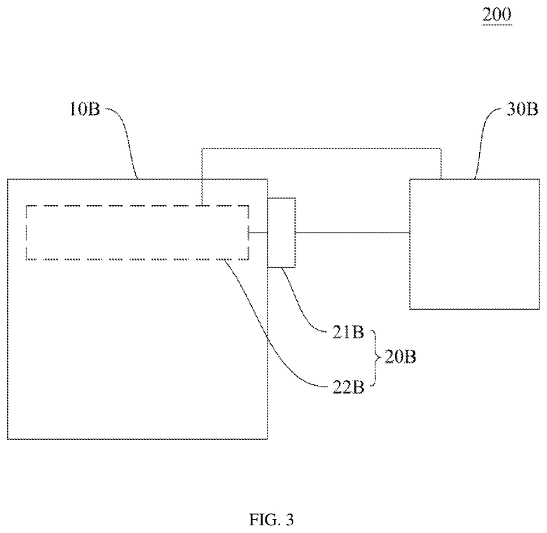

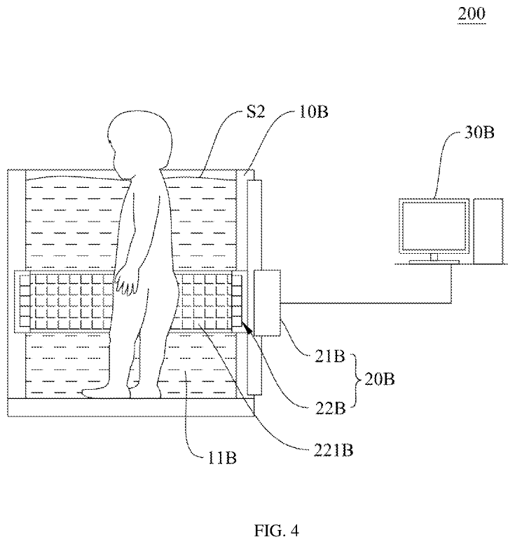

The embodiment detailed below is different from the previous one mainly in the configuration of the ultrasound probe array. The remaining aspects of the following embodiment are identical to those of the foregoing embodiment and therefore will not be described repeatedly. Please refer now to FIG. 3 and FIG. 4 respectively for a block diagram and a schematic drawing of the second embodiment of the invention.

The second embodiment discloses an ultrasound 3D full-body tomography system 200, which includes an ultrasound guided wave medium container 10B, a movable ultrasound probe assembly 20B, and an ultrasound tomography device 30B.

The ultrasound guided wave medium container 10B has a detection space S2. The detection space S2 is filled with a guided wave medium 11B so that a subject's body can be immersed in the guided wave medium 11B. In one preferred embodiment, the ultrasound guided wave medium container 10B is provided with an acoustic insulation layer made of a sound-absorbing or noise-reducing material. As a barrier between the movable ultrasound probe assembly 20B and the environment outside the system, the acoustic insulation layer may be provided anywhere on/in the ultrasound guided wave medium container 10B (e.g., on the outer or inner wall of the container or inside the container) as long as the acoustic insulation layer corresponds in position to the movable ultrasound probe assembly 20B. To produce satisfactory detection results, the guided wave medium 11B may be water, degassed water, a developing agent, or a wave guiding gel; the present invention has no limitation in this regard.

The movable ultrasound probe assembly 20B includes a linear carrier 21B and an annular ultrasound probe array 22B provided on the linear carrier 21B. The annular ultrasound probe array 22B includes a plurality of probe units 221B, which are integrated into an annular array. The annular ultrasound probe array 22B can be moved back and forth along the detection space S2 via the linear carrier 21B. To construct a 3D model, the annular ultrasound probe array 22B sends the velocity of the linear carrier 21B to the ultrasound tomography device 30B in addition to the three types of parameters mentioned above, namely the location of each probe unit 221B that has received an echo (i.e., the location of each response-receiving pixel of the array), the signal intensity of each echo, and the flight times of ultrasonic waves (i.e., the response times). The velocity of the linear carrier 21B serves as a basis on which to correct the reported locations of the response-receiving probe units 221B.

The ultrasound tomography device 30B is an image processing device configured to construct a 3D image model based on the data fed back from each pixel of the annular ultrasound probe array 22B. As mentioned above, the ultrasound tomography device 30B receives four major types of parameters from the annular ultrasound probe array 22B: the location of each probe unit 221B that has received an echo (i.e., the location of each response-receiving pixel of the array), the velocity of the linear carrier, the signal intensity of each echo, and the flight times of ultrasonic waves (i.e., the response times).

According to the above, the present invention uses an array of ultrasound probes to perform 3D full-body imaging on a patient, thereby enabling multidimensional image reconstruction for tissues that are impenetrable by ultrasonic waves. The prior art drawback of insufficient imaging is thus overcome. In addition, according to the present invention, 3D full-body imaging is performed on a patient through an array of ultrasound probes to effectively prevent injury to the human body. Furthermore, according to the present invention, the array of ultrasound probes can directly output 3D images of a patient or a body part, without having to convert 2D images into 3D ones in a subsequent step.

The above is the detailed description of the present invention. However, the above is merely the preferred embodiment of the present invention and cannot be the limitation to the implement scope of the present invention, which means the variation and modification according to the present invention may still fall into the scope of the invention.

What is claimed is:

1. An ultrasound three-dimensional (3D) full-body tomography system, comprising:
an ultrasound guided wave medium container having a detection space, wherein the detection space is filled with a guided wave medium in order for a subject's body to be immersed in the guided wave medium;
an ultrasound probe array provided in the ultrasound guided wave medium container and comprising a plurality of probe units, wherein the probe units are integrated into an annular array and are arranged along a periphery of the detection space; and
an ultrasound tomography device for constructing a 3D image model based on data fed back from each pixel of the ultrasound probe array.

2. The ultrasound 3D full-body tomography system of claim 1, wherein the guided wave medium is water, degassed water, a developing agent, or a wave guiding gel.

3. The ultrasound 3D full-body tomography system of claim 1, wherein the ultrasound guided wave medium container is provided with an acoustic insulation layer.

4. An ultrasound three-dimensional (3D) full-body tomography system, comprising:
an ultrasound guided wave medium container having a detection space, wherein the detection space is filled with a guided wave medium in order for a subject's body to be immersed in the guided wave medium;
a movable ultrasound probe assembly comprising a linear carrier and an annular ultrasound probe array provided on the linear carrier, wherein the annular ultrasound probe array comprises a plurality of probe units integrated into an annular array and is movable along the detection space via the linear carrier; and
an ultrasound tomography device for constructing a 3D image model based on a velocity of the linear carrier and on data fed back from the annular ultrasound probe array.

5. The ultrasound 3D full-body tomography system of claim 4, wherein the guided wave medium is water, degassed water, a developing agent, or a wave guiding gel.

6. The ultrasound 3D full-body tomography system of claim 4, wherein the ultrasound guided wave medium container is provided with an acoustic insulation layer.
